(19) United States
(12) Patent Application Publication
(10) Pub. No.: US 2024/0358227 A1
(43) Pub. Date: Oct. 31, 2024

(54) MEDICAL VALVE ASSEMBLIES AND RELATED METHODS THEREOF

(71) Applicant: Boston Scientific Scimed, Inc., Maple Grove, MN (US)

(72) Inventor: Alexander James LAMMERS, Boston, MA (US)

(73) Assignee: Boston Scientific Scimed, Inc., Maple Grove, MN (US)

(21) Appl. No.: 18/645,589

(22) Filed: Apr. 25, 2024

Related U.S. Application Data
(60) Provisional application No. 63/498,650, filed on Apr. 27, 2023.

(51) Int. Cl. A61B 1/00; A61B 1/015; A61B 1/12

(52) U.S. Cl. CPC A61B 1/00068; A61B 1/015; A61B 1/126

(57) ABSTRACT

A handle assembly for a medical device, including a handle body, a first valve body housed at least partially within the handle body, a second valve body housed at least partially within the handle body and positioned adjacent to the first valve body, a lens wash inlet port extending from a first side the second valve body at a first angle with respect to a longitudinal axis of the second valve body, and a lens wash outlet port extending from a second side of the second valve body at a second angle with respect to the longitudinal axis of the second valve body. The first side of the second valve body faces a first direction and the second side of the second valve body faces a second direction, where the first direction is substantially opposite to the second direction.

### CROSS-REFERENCE TO RELATED APPLICATIONS

This application claims the benefit of priority under 35 U.S.C. § 119 from U.S. Provisional Application No. 63/498,650, filed Apr. 27, 2023, which is incorporated by reference herein in its entirety.

### TECHNICAL FIELD

Various aspects of this disclosure relate generally to medical valve assemblies, devices, and related methods thereof. More specifically, embodiments of the disclosure relate to valve bodies and associated structures within the handles of scope devices.

### BACKGROUND

Medical devices, particularly scope devices such as duodenoscopes, endoscopes, ureteroscopes, bronchoscopes, cystoscopes, colonoscopes, laparoscopes, and laryngoscopes, typically include handle assemblies, which house components and control mechanisms of the devices. For example, handle assemblies of scope devices may include fluidics components, such as valve bodies that receive valves for delivering fluids (e.g., air and/or water) or suction to a distal tip of the scope device, along with associated tubing. The handle assembly may also house numerous other elements (e.g., steering controls, electronics, working channels, and/or ports). The fluidics components within the interiors of the handle assemblies may occupy a large portion of the interior handle space, which may limit the space available to other components which may be incorporated into the handle assembly. Furthermore, the fluidics components (e.g., tubing) may create a tortuous path for fluid to pass through, which may restrict the flow of fluid. Therefore, a need exists for medical valve assemblies, devices, and related methods thereof.

### SUMMARY

Each of the aspects disclosed herein may include one or more of the features described in connection with any of the other disclosed aspects.

Aspects of the disclosure relate to, among other things, systems, devices, and methods for handle assemblies for medical devices, for example handle assemblies of scope devices including valves and tubing.

According to an example, a handle assembly for a medical device may include a handle body a first valve body housed at least partially within the handle body, a second valve body housed at least partially within the handle body and positioned adjacent to the first valve body, a lens wash inlet port extending from a first side the second valve body at a first angle with respect to a longitudinal axis of the second valve body, and a lens wash outlet port extending from a second side of the second valve body at a second angle with respect to the longitudinal axis of the second valve body. The first side of the second valve body may face a first direction and the second side of the second valve body may face a second direction, where the first direction is substantially opposite to the second direction.

Any of the handle assemblies described herein may include any of the following features. The handle assembly may include an insufflation inlet port extending from the first side of the second valve body at a third angle with respect to the longitudinal axis of the second valve body, and an insufflation outlet port extending from the second side of the second valve body at a fourth angle with respect to the longitudinal axis of the second valve body. The first angle, the second angle, the third angle, and the fourth angle are equal. The first angle, the second angle, the third angle, and the fourth angle are each approximately 45°. The first angle, the second angle, and the third angle are approximately equal, and the fourth angle is different from each of the first angle, the second angle, and the third angle. Each of the first angle, the second angle, and the third angle is approximately 90°, and the fourth angle is approximately 45°. The lens wash outlet port extends toward a first end of the second valve body, and each of the insufflation outlet port, the insufflation inlet port, and the lens wash inlet port extend toward a second end of the second valve body. An interface between the insufflation outlet port and an inner chamber of the second valve body is positioned closer to a first end of the second valve body with respect to each of the insufflation inlet port, the lens wash inlet port, and the lens wash outlet port. An interface between the lens wash outlet port and a well of the second valve body is positioned closer to a first end of the second valve body with respect to the lens wash inlet port, and closer to a second end of the second valve body with respect to each of the insufflation inlet port and the insufflation outlet port. The first valve body includes a suction inlet port and a suction outlet port, and the suction outlet port is positioned closer to a first end of the first valve body with respect to the suction inlet port. The suction outlet port extends towards the second valve body. The lens wash outlet port and the lens wash inlet port are collinear. A longitudinal axis of the first valve body is parallel to an axis of the handle body, and the longitudinal axis of the second valve body is at an angle with respect to the axis of the handle body. The angle of the second valve body with respect to the axis of the handle body is in a range of 0° to 45°. Each of the longitudinal axis of the second valve body and a longitudinal axis of the first valve body are parallel to an axis of the handle body.

According to another example, a handle assembly for a medical device may include a handle body, a first valve body housed at least partially within the handle body, and a second valve body housed at least partially within the handle body and positioned adjacent to the first valve body, where a longitudinal axis of the first valve body is parallel to an axis of the handle body, and where a longitudinal axis of the second valve body is at an angle with respect to the longitudinal axis of the handle body.

Any of the handle assemblies described herein may include any of the following features. The angle of the longitudinal axis of the second valve body is in a range of 0° to 45°. The handle assembly may include a lens wash inlet port extending from a first side the second valve body at a first angle with respect to a longitudinal axis of the second valve body, a lens wash outlet port extending from a second side of the second valve body at a second angle with respect to the longitudinal axis of the second valve body, an insufflation inlet port extending from the first side of the second valve body at a third angle with respect to the longitudinal axis of the second valve body, and an insufflation outlet port extending from the second side of the second valve body at a fourth angle with respect to the longitudinal axis of the second valve body. The lens wash inlet port and the lens wash outlet port are collinear.

According to another example, a handle assembly for a medical device may include a handle body, a first valve body housed at least partially within the handle body, a second valve body housed at least partially within the handle body and positioned adjacent to the first valve body, a lens wash inlet port extending from a first side the second valve body, toward a first end of the second valve body and at a first angle with respect to a longitudinal axis of the second valve body, and a lens wash outlet port extending from a second side of the second valve body, toward a second end of the second valve body and at a second angle with respect to the longitudinal axis of the second valve body. The first angle may be equal to the second angle, a longitudinal axis of the first valve body may be parallel to an axis of the handle body, and a longitudinal axis of the second valve body may be at an angle with respect to the longitudinal axis of the handle body, where the lens wash inlet port and the lens wash outlet port are collinear

It may be understood that both the foregoing general description and the following detailed description are exemplary and explanatory only and are not restrictive of the invention, as claimed. As used herein, the terms “comprises,” “comprising,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “top” refers to a direction or side of a device relative to its orientation during use, and the term “bottom” refers to a direction or side of a device relative to its orientation during use that is opposite of the “top.” The term “distal” refers to a direction away from an operator/toward a treatment site, and the term “proximal” refers to a direction toward an operator. The term “exemplary” is used in the sense of “example,” rather than “ideal.” The term “approximately,” or like terms (e.g., “substantially”), includes values +/−10% of a stated value.

### BRIEF DESCRIPTION OF THE DRAWINGS

The accompanying drawings, which are incorporated in and constitute a part of this specification, illustrate aspects of this disclosure and together with the description, serve to explain the principles of the disclosure.

FIG. 1 shows a cross-sectional side view of a handle assembly of a medical device according to some embodiments.

FIG. 2 shows a cross-sectional side view of a valve body according to some embodiments.

FIG. 3 shows a cross-sectional side view of a handle assembly of a medical device according to some embodiments.

FIG. 4 shows a cross-sectional side view of a valve body according to some embodiments.

### DETAILED DESCRIPTION

Embodiments of this disclosure relate to medical devices, particularly scope devices such as duodenoscopes, which typically include handle assemblies having a variety of valves and conduits (e.g., tubing) used to circulate gases, liquids, and/or suction through the devices. The fluidic valves and tubes are typically housed within the interiors of the handle assemblies and often occupy a large portion, for example approximately 50%, of the interior handle space. In some examples, as the dimensions of the valve bodies used to house the valves may be dictated the specific valves that are desired to be used with the medical devices. In conventional arrangements, the tubing may have a tortuous arrangement, due to the length of the tubes in combination with space limitations, which may cause a restriction of the flow of fluid through the tubing. The amount of space occupied by the valves and the tubing may also limit the number of components which may be incorporated into the handle.

The presently disclosed embodiments include a valve body for receiving a valve stem, such as an air/water valve stem. The valve body may include two inlets, one for each of air and water (or other fluids). The valve body may further include two outlets, one for each of air and water (or other fluids). In comparison to conventional valve bodies, in which the two inlets and two outlets are on the same side of the valve body (e.g., a side of the valve body facing away from a shaft of the medical device, such as a proximal direction), the presently disclosed embodiments may include inlets and outlets on different sides of the valve body. For example, the two inlets may be located on a first side of the valve body (e.g., a side of the valve body facing away from the shaft of the medical device and in a direction toward an umbilicus of the medical device), and the two outlets may be located on a second side of the valve body (e.g., a side facing the shaft of the medical device). The inlets and/or outlets may have orientations (e.g., angles) to accommodate other components, to aid in streamlining manufacturing and/or to offer a more direct path for the fluid to flow. The valve bodies may accommodate existing valve stems without modifications to the valve stem.

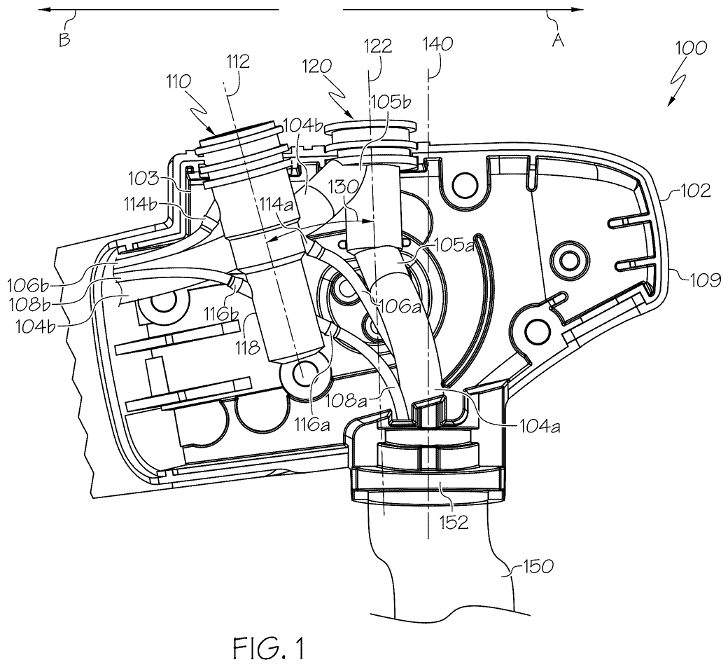

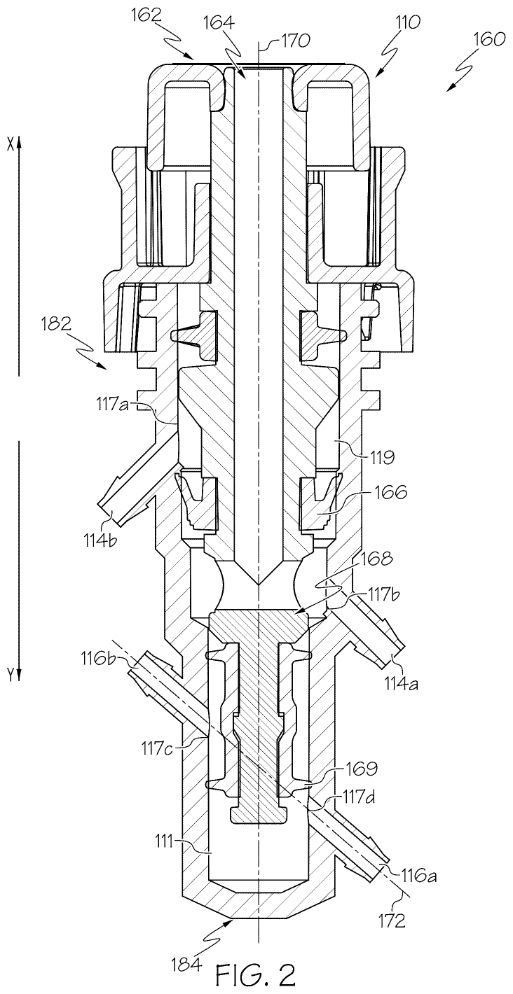

FIG. 1 shows a cross-sectional side view of a space-saving handle assembly according to some embodiments. For example, a handle assembly 100 may include a handle body 102 configured to house at least an air/water (AW) valve body 110 positioned adjacent to a suction valve body 120, suction inlet tubing 104a, suction outlet tubing 104b, insufflation (i.e., air) inlet tubing 106a, insufflation outlet tubing 106b, lens wash (i.e., water) inlet tubing 108a, and lens wash outlet tubing 108b. FIG. 2 shows details of an assembly 160 including AW valve body 110 and a valve 162 received therein. Although air and water are referred to herein, it will be appreciated that AW valve body 110 and tubing 104a, b and 106a, b may alternatively serve to deliver alternative fluids. AW valve body 110 may receive (e.g., removably receive) an AW valve 162, which may be coupled via AW valve body 110 to each of insufflation tubing 106a, b and lens wash tubing 108a, b. The AW valve may function to selectively supply a medical device, for example a distal tip of a duodenoscope, with air and water during a medical procedure. Similarly, suction valve body 120 may receive (e.g., removably receive) a suction valve (not shown), which may be coupled to suction inlet tubing 104a and suction outlet tubing 104b via suction valve body 120. The suction valve may function to provide suction to a medical device, for example a distal tip of a duodenoscope, during a medical procedure via a vacuum source. For example, fluids suctioned from the distal tip of the medical device may flow proximally from the distal tip, through suction outlet tubing 104b, to suction valve body 120. The suctioned fluids may flow only proximally; in other words, at no portion of suction outlet tubing 104b may fluids suctioned from the distal tip travel distally. Suction inlet tubing 104a, insufflation inlet tubing 106a, and lens wash inlet tubing 108a each may extend from respective sources of suction, insufflation (air), and lens wash (water), through an umbilicus 150, and into handle body 102 via a coupling section 152 of handle body 102 that couples handle body 102 to umbilicus 150. The suction inlet tubing 104a, insufflation inlet tubing 106a, and lens wash inlet tubing 108a may extend through the coupling section 152 to AW valve body 110 (insufflation inlet tubing 106a and lens wash inlet tubing 108a) and suction valve body 120 (suction inlet tubing 104a). Insufflation inlet tubing 106a may couple to an insufflation inlet port 114a of AW valve body 110. Lens wash inlet tubing 108a may couple to a lens wash inlet port 116a of AW valve body 110. Suction inlet tubing 104a may couple to a suction inlet port 105a of suction valve body 120.

Insufflation outlet tubing 106b and lens wash outlet tubing 108b may extend distally from AW valve body 110, through handle body 102, into a shaft (not shown) of the medical device, and distally through the shaft to a distal tip of the shaft, Suction outlet tubing 104b may similarly extend distally from suction valve body 120, through handle body 102, into the shaft, and distally through the shaft to the distal tip of the shaft. Insufflation outlet tubing 106b may couple to an insufflation outlet port 114b of AW valve body 110. Lens wash outlet tubing 108b may couple to a lens wash outlet port 116b of AW valve body 110. Suction outlet tubing 104b may couple to a suction outlet port 105b of suction valve body 120.

As mentioned above, in conventional AW valve bodies, all insufflation inlet and outlet ports and lens wash inlet and outlet ports may be on a same side of an AW valve body (e.g., facing away from an insertable shaft of the medical device). In contrast, as shown in FIGS. 1 and 2, insufflation inlet port 114a and lens wash inlet port 116a may be on a first side of AW valve body 110, and insufflation outlet port 114b and lens wash outlet port 116b may be on a substantially opposite side of AW valve body 110. Inlet ports 114a, 116a be on a side of valve body 110 facing direction A of FIG. 1 (i.e., facing proximally toward a rear wall 109 of handle body 102, and away from an insertable shaft of the medical device). Outlet ports 114b, 116b may be on a side of valve body 110 facing direction B of FIG. 1 (i.e., facing toward a distal end of handle body and toward the insertable shaft of the medical device).

As shown in FIG. 1, insufflation outlet tubing 106b and lens wash outlet tubing 108b may extend distally from the AW valve body 110 without any portions of insufflation outlet tubing 106b or lens wash outlet tubing 108b extending proximally (i.e., with a component along direction A, toward rear wall 109) from the AW valve body 110. In other words, a flow of fluids through insufflation outlet tubing 106b and lens wash outlet tubing 108b may not be in a proximal direction or partially proximal direction at any portion of insufflation outlet tubing 106b and lens wash outlet tubing 108b. This contrasts with conventional AW valve bodies in which outlet ports for insufflation and lens wash are on a same, side of the valve bodies, facing proximally away from the insertable shaft of the medical device. In those conventional AW valve assemblies, the insufflation and lens wash outlet tubes include portions that extend at least somewhat proximally, such that fluid flows at least partially in a proximal direction along portions of the outlet tubes. As discussed above, AW valve body 110 may be dimensioned to accommodate a specific AW valve. AW valve body 110 may receive and operate with a same AW valve as a conventional valve body having the outlet ports facing the proximal side of the handle body.

As shown in FIG. 1 and particularly in FIG. 2, AW valve body 110 may have insufflation ports 114a, 114b and lens wash ports 116a, 116b positioned at an angle with respect to a longitudinal axis 170 of AW valve body 110. As used herein, a first end 182 of valve body 110 is toward a first direction (labeled X of FIG. 2), and a second end 184 of valve body 110 is toward a second direction (labeled Y in FIG. 2). The first direction (direction X) is toward where a user's finger would contact valve 162, and the second direction (direction Y) is away from where the user's finger would contact valve 162. An interface 117a between insufflation outlet port 114b and an inner chamber 119 of AW valve body 110 may be positioned closer to first end 182 of valve body 110 with respect to each of insufflation inlet port 114b, lens wash inlet port 116a, and lens wash outlet port 116b. An interface 117b between insufflation inlet port 114a and inner chamber 119 may be positioned closer to second end 184 of valve body 110 with respect to insufflation outlet port 114b, and closer to first end 182 of valve body 110 with respect to each of lens wash inlet port 116a and lens wash outlet port 116b. An interface 117c between lens wash outlet port 116b and a well 111 of AW valve body 110 may be positioned closer to second end 184 of valve body 110 with respect to each of insufflation inlet port 114a and insufflation outlet port 114b, and closer to first end 182 of valve body 110 with respect to lens wash inlet port 116a. Finally, an interface 117d between lens wash inlet port 116a and well 111 may be positioned closer to second end 184 of valve body 110 with respect to each of insufflation outlet port 114b, insufflation inlet port 114a, and lens wash outlet port 116b.

As shown in FIG. 2, each of insufflation inlet port 114a, insufflation outlet port 114b, lens wash inlet port 116a, and lens wash outlet port 116b may be positioned at an angle with respect to a longitudinal axis 170 of AW valve body 110. In some embodiments, each of ports 114a, 114b, 116a, 116b may be positioned at an equal angle with respect to longitudinal axis 170. For example, each of ports 114a, 114b, 116a, and 116b may be positioned at an approximately 45° angle with respect to longitudinal axis 170. Each of insufflation inlet port 114a, insufflation outlet port 114b, and lens wash inlet port 116a may extend partially in the second direction (direction Y) from AW valve body 110 at an approximately 45° angle with respect to longitudinal axis 170. Lens wash outlet port 116b may extend partially in the first direction (direction X) from AW valve body 110 at an approximately 45° angle with respect to longitudinal axis 170. Lens wash inlet port 116a and lens wash outlet port 116b may be positioned collinearly, such that each of lens wash inlet port 116a and lens wash outlet port 116b extend along an axis 172, which may extend transversely to longitudinal axis 170 (e.g., at an approximately 45° angle to longitudinal axis 170).

The collinear positioning of lens wash inlet port 116a and lens wash outlet port 116b may allow for a streamlined tubing path for lens wash tubing, as the tubing may be positioned within a handle body without excessive bending, which, as discussed above, improve the flow rate of the fluid through the tubing. The collinear positioning of ports 116a and 116b may also aid in increasing the ease of manufacturability of AW valve body 110, as a boring tool may make a single pass to create openings in both of ports 116a and 116b. Furthermore, because the collinear positioning of ports 116a and 116b may aid in the avoidance of excessive bending of the tubing, strain-relief components used to support the tubing, such as springs, may not be necessary. The option to exclude such strain-relief components may aid in decreasing manufacturing and assembly complexity.

As discussed above, insufflation outlet port 114b may extend along directions B and Y of FIGS. 1-2 (toward the insertable shaft of the medical device, and away from first end 182 of valve body 110). This may prevent insufflation outlet port 114b and/or insufflation outlet tubing 106b from contacting a sidewall 103 of handle body 102. This prevents restriction of the flow of fluid through the insufflation outlet tubing 106b. Angles of inlet ports 114a, 116b (along A and Y directions of FIGS. 1-2, i.e., away from the insertable shaft and away from first end 182 of valve body 110) may prevent inlet tubing 106a, 108a from contacting suction valve body 120.

Somewhat similarly, in conventional suction valve bodies, a suction outlet port may be disposed on a substantially proximal side of the suction valve body. Thus, outlet tubing extending from a conventional suction valve body may include a portion that extends at least partially in a proximal direction (e.g., at an angle that has a proximal component). In contrast, suction outlet port 105b of suction valve body 120 may be disposed on a substantially distal side of suction valve body 120. Suction outlet tubing 104b may extend distally without any portions of suction outlet tubing 104b extending proximally. As discussed above, fluids suctioned from the distal tip of the medical device may flow proximally from the distal tip, through suction outlet tubing 104b, to suction valve body 120. in these aspects, the suctioned fluids may flow proximally; in other words, at no portion of suction outlet tubing 104b may fluids suctioned from the distal tip travel distally. Such an arrangement contrasts with conventional suction valve bodies and tubing. As discussed above, suction valve body 120 may be dimensioned to accommodate a specific suction valve. Suction valve body 120 may receive and operate with a same suction valve as a conventional valve body having the outlet port on the proximal side of the conventional valve body. As shown in FIG. 1, suction outlet port 105b may extend somewhat into the page of FIG. 1, away from steering pulleys of handle assembly 100, thereby directing suction outlet tubing 104b behind AW valve body 110. In contrast, in conventional arrangements, an outlet port of a suction valve body may extend somewhat toward steering pulleys of the handle assembly.

As shown in FIG. 1, a longitudinal axis 112 of AW valve body 110 may be positioned at a first angle with respect to a handle axis extending centrally through coupling section 152 and a portion of umbilicus 150 just distal of coupling section 152 (a vertical direction of FIG. 1). A longitudinal axis 122 of suction valve body 120 may be positioned at a second angle with respect to handle axis 140. Specifically, longitudinal axis 112 of AW valve body 110 may be positioned at an angle in a range of 0° to 45°, including subranges, with respect to handle axis 140, while a longitudinal axis 122 of suction valve body 120 may extend substantially parallel to longitudinal axis 140. Accordingly, valve body 110 may be positioned with respect to valve body 120 at an angle 130. In some embodiments, angle 130 may be in a range of 0° to 45°, including subranges.

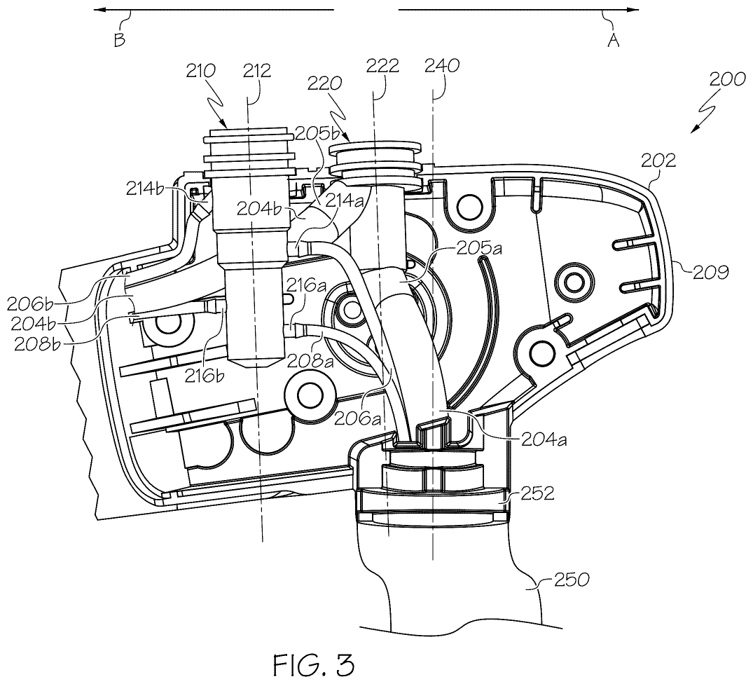

Positioning AW valve body 110 at angle 130 may save space within handle body 102, as compared to an arrangement in which axes 112 and 122 are substantially parallel to one another (as in FIG. 3, discussed in further detail below). In some embodiments, as discussed in further detail below, the angled positioning of AW valve body 110 may also allow for streamlined positioning of both insufflation tubing 106a, b and of lens wash tubing 108a, b. For example, by positioning AW valve body 110 at an angle, the area between a wall-facing face 118 of AW valve body 110 and sidewall 103 of handle body 102 may be increased, which may facilitate positioning of insufflation outlet port 114b and lens wash outlet port 116b to be positioned on wall-facing face 118 (i.e., on a proximal side of AW valve body 110), as discussed above.

Additionally, in some embodiments, positioning AW valve body 110 at angle 130 may help to enhance ergonomics of handle body 102. For example, during use, an operator may position two of his or her fingers on an AW valve positioned in AW valve body 110 and a suction valve positioned in suction valve body 120. The angled placement of AW valve body 110 may allow for the fingers to be positioned in a more comfortable position as compared to an AW valve body that is substantially parallel to a suction valve body, which may contribute to a reduction in user fatigue during a medical procedure.

Referring to FIGS. 1-2, in operation, when a user covers an opening 164 on a first end (toward direction X) of valve 162 (FIG. 2), air may flow from insufflation inlet tubing 106a, through insufflation inlet port 114a, along the first direction (direction X) past a one-way seal 166 positioned around a valve stem 168 of valve 162, and through insufflation outlet port 116a, to insufflation outlet tubing 106b. As shown in FIG. 2, valve 162 may be in a configuration such that water (lens wash) is not delivered. Upon depressing valve 162, a seal 169 around valve stem 168 moves along the second direction (Y direction) closer to second end 184 of AW valve body 110 until seal 169 is positioned between second end 184 and interface 117d between lens wash inlet port 116a and well 111 of AW valve body 110. Water (lens wash) may flow from lens wash inlet tubing 108a, through lens wash inlet port 116a, along an outer surface of valve stem 168, through lens wash outlet port 116b, and into lens wash outlet tubing 108b. While water is being delivered, one or more seals of valve 162 may prevent delivery of air/insufflation.

Alternatively, for example as shown in FIG. 3, a handle assembly 200 may include an AW valve body 210 and a suction valve body 220 disposed substantially parallel to one another within a handle body 202.

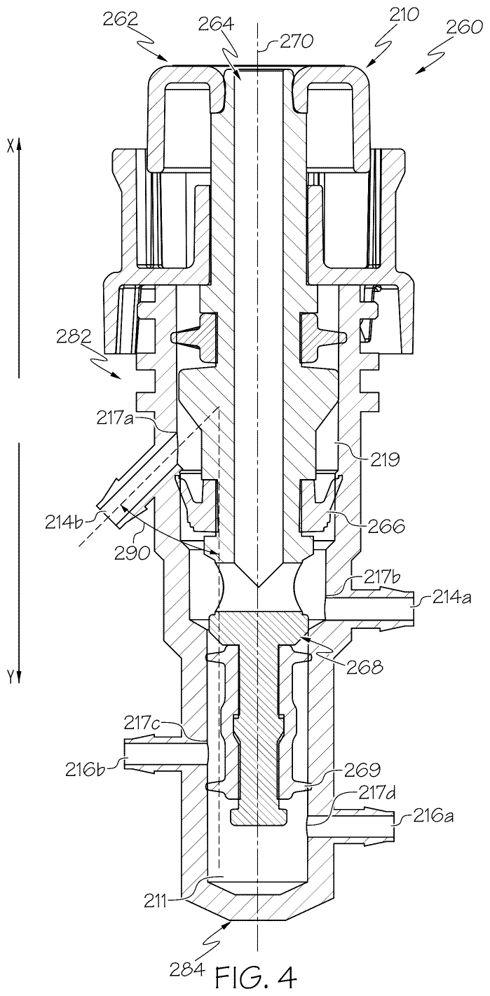

Similar to handle assembly 100, described above, handle assembly 200 may include handle body 202, which may be configured to house at least AW valve body 210, suction valve body 220, suction inlet tubing 204a, suction outlet tubing 204b, insufflation inlet tubing 206a, insufflation outlet tubing 206b, lens wash inlet tubing 208a, and lens wash outlet tubing 208b. Handle assembly 200 may have any of the properties of handle assembly 100, except as specified herein. FIG. 4 shows details of an assembly 260 including AW valve body 210 and a valve 262 received therein. Valve 262 may have any of the properties of valve 162, except as specified herein. Although air and water are referred to herein, it will be appreciated that AW valve body 210 and tubing 206a, b and 208a, b may alternatively serve to deliver alternative fluids.

AW valve body 210 may receive (e.g., removably receive) an AW valve 262, which may be coupled via AW valve body 210 to each of insufflation tubing 206a, b and lens wash tubing 208a, b. As with valve 162, AW valve 262 may function to selectively supply a medical device, for example a distal tip of a duodenoscope, with air and water during a medical procedure. Similarly, suction valve body 220 may receive (e.g., removably receive) a suction valve (not shown), which may be coupled to suction inlet tubing 204a and suction outlet tubing 204b via suction valve body 220. The suction valve may function to provide suction to a medical device, for example a distal tip of a duodenoscope, during a medical procedure via a vacuum source.

Similar to handle assembly 100, discussed above, in some embodiments, suction inlet tubing 204a, insufflation inlet tubing 206a, and lens wash inlet tubing 208a each may extend from respective sources of suction, insufflation (air), and lens wash (water), through an umbilicus 250, and into handle body 202 via a coupling section 252 of handle body 202 that couples handle body 202 to umbilicus 250. Suction inlet tubing 204a, insufflation inlet tubing 206a, and lens wash inlet tubing 208a may extend through the coupling section 252 to AW valve body 210 (insufflation inlet tubing 206a and lens wash inlet tubing 208a) and suction valve body 220 (suction inlet tubing 204a). Insufflation inlet tubing 206a may couple to an insufflation inlet port 214a of AW valve body 210. Lens wash inlet tubing 208a may couple to a lens wash inlet port 216a of AW valve body 210. Suction inlet tubing 204a may couple to a suction inlet port 205a of suction valve body 220.

Insufflation outlet tubing 206b and lens wash outlet tubing 208b may extend distally from AW valve body 210, through handle body 202, into a shaft (not shown) of the medical device, and distally through the shaft to a distal tip of the shaft, Suction outlet tubing 204b may similarly extend distally from suction valve body 220, through handle body 202, into the shaft, and distally through the shaft to the distal tip of the shaft. Insufflation outlet tubing 206b may couple to an insufflation outlet port 214b of AW valve body 110. Lens wash outlet tubing 208b may couple to a lens wash outlet port 216b of AW valve body 210. Suction outlet tubing 204b may couple to a suction outlet port 205b of suction valve body 220.

Like AW valve body 110, discussed above, insufflation inlet port 214a and lens wash inlet port 216a may be on a first side of AW valve body 210, and insufflation outlet port 214b and lens wash outlet port 216b may be on a second, opposite side of AW valve body 210. Inlet ports 214a, 216a be on a side of valve body 110 facing direction A of FIG. 3 (i.e., facing proximally toward a rear wall 209 of handle body 202, and away from an insertable shaft of the medical device). Outlet ports 214b, 216b may be on a side of valve body 210 facing direction B of FIG. 3 (i.e., facing toward a distal end of handle body and toward the insertable shaft of the medical device). Additionally, insufflation outlet tubing 206b and lens wash outlet tubing 208b may extend distally from AW valve body 210 without any portions of insufflation outlet tubing 206b or lens wash outlet tubing 208b extending proximally (i.e., with a component along direction A, toward rear wall 209) from the AW valve body 210.

Additionally, for example as shown in FIG. 4, AW valve body 210 may have insufflation ports 214a, 214b and lens wash ports 216a, 216b. As used herein, a first end 282 of valve body 210 is toward a first direction (labeled X of FIG. 4), and a second end 284 of valve body 210 is toward a second direction (labeled Y in FIG. 4). The first direction (direction X) is toward where a user's finger would contact valve 262, and the second direction (direction Y) is away from where the user's finger would contact valve 262.

In some embodiments, an interface 217a between insufflation outlet port 214b and an inner chamber 219 of AW valve body 210 may be positioned closer to first end 282 of valve body 210 with respect to each of insufflation inlet port 214a, lens wash inlet port 216a, and lens wash outlet port 216b. An interface 217b between insufflation inlet port 214a and inner chamber 219 may be positioned closer to second end 284 of valve body 210 with respect to insufflation outlet port 214b, and closer to first end 282 of valve body 210 with respect to each of lens wash inlet port 216a and lens wash outlet 216b. An interface 217c between lens wash outlet port 216b and a well 211 of AW valve body 210 may be positioned closer to the second end 284 of valve body 210 with respect to each of insufflation inlet port 214a and insufflation outlet port 214b, and closer to first end 282 of valve body 210 with respect to lens wash inlet port 216a. Finally, an interface 217d between lens wash inlet port 216a and well 211 may be positioned closer to second end 284 of valve body 210 with respect to each of insufflation outlet port 214b, insufflation inlet port 214a, and lens wash outlet port 216b

Furthermore, similarly to handle 100, suction outlet port 205b of suction valve body 220 may be disposed on a substantially distal side of suction valve body 220. Suction outlet tubing 204b may extend distally without any portions of suction outlet tubing 204b extending proximally. Fluids suctioned from a distal tip of the medical device may flow proximally from the distal tip, through suction outlet tubing 204b, to suction valve body 220. Suction valve body 220 may receive and operate with a same suction valve as a conventional valve body having the outlet port on the proximal side of the conventional valve body. As shown in FIG. 3, suction outlet port 205b may extend somewhat into the page of FIG. 3, away from steering pulleys of handle assembly 200 thereby directing suction outlet tubing 204b behind AW valve body 110.

In contrast to handle assembly 100, however, a longitudinal axis 212 of AW valve body 210 may be substantially parallel to a longitudinal axis 222 of suction valve body 220 and to a handle axis 240 of handle assembly 200. Longitudinal axis 212 may be approximately perpendicular to the directions A and B of FIG. 3. Accordingly, the configurations of valve bodies 210 and 220 may closely mirror the configurations of AW and suction valve bodies in existing handle assembly designs, which may allow for more efficient and more cost-effective manufacturing. Furthermore, such an arrangement may provide familiar ergonomics to users. In such embodiments, to aid in reducing the amount of space occupied by the fluidics within handle body 202, the positions of the inlet and outlet ports on valve bodies 210 and 220 may be adjusted, for example as shown in FIG. 4.

In some embodiments, each of insufflation inlet port 214a, lens wash inlet port 216a, and lens wash outlet port 216b may extend outwardly from AW valve body 210 at an approximately equal angle with respect to a longitudinal axis 270 of AW valve body 210. For example, each of insufflation inlet port 214a, lens wash inlet port 216a, and lens wash outlet port 216b may extend outwardly from AW valve body 210 at an angle of approximately 90° with respect to a longitudinal axis 270 of AW valve body 210. However, insufflation outlet port 214b may extend partially in the second direction (direction Y) away from AW valve body 210 at an angle 290 with respect to longitudinal axis 270. Angle 310 may be in a range of 0° to 90°, including subranges. In some embodiments, angle 290 may be approximately 45°. By positioning insufflation outlet port 214b at angle 290, AW valve body 210 may be incorporated into a handle assembly designed to house both an AW valve body and a suction valve body at a parallel position with respect to a longitudinal axis of the handle assembly, for example handle assembly 200, while avoiding contact between a sidewall of the handle assembly and an insufflation tube extending from insufflation outlet port 214b, for example insufflation outlet tubing 206b. Such arrangement of insufflation outlet tubing 206b is discussed above, with respect to handle 100. Angling of insufflation outlet port 214b may aid in the positioning of an insufflation tube to help avoid restriction of fluid through the tube.

Referring to FIGS. 3-4, in operation, when a user covers an opening 264 on a first end (toward direction X) of valve 262 (FIG. 4), air may flow from insufflation inlet tubing 206a, through insufflation inlet port 214a, along the first direction (direction X) past a one-way seal 266 positioned around a valve stem 268 of valve 262, and through insufflation outlet port 216a, to insufflation outlet tubing 206b. As shown in FIG. 4, valve 262 may be in a configuration such that water (lens wash) is not delivered. Upon depressing AW valve 262, a seal 269 around valve stem 268 moves along the second direction (Y direction) closer to second end 284 of AW valve body 210 until seal 269 is positioned between second end 284 and interface 217d between lens wash inlet port 216a and well 211 of AW valve body 210. Water (lens wash) may flow from lens wash inlet tubing 208a, through lens wash inlet port 216a, along an outer surface of valve stem 268, through lens wash outlet port 216b, and into lens was outlet tubing 208b. While water is being delivered, one or more seals of valve 262 may prevent delivery of air/insufflation.

While principles of this disclosure are described herein with reference to illustrative examples for particular applications, it should be understood that the disclosure is not limited thereto. For example, the disclosure refers to duodenoscopes as an exemplary scope including a handle assembly. The systems, devices, and methods of the present disclosure, however, may be used in any suitable scope device. Those having ordinary skill in the art and access to the teachings provided herein will recognize additional modifications, applications, and substitution of equivalents all fall within the scope of the examples described herein. Accordingly, the invention is not to be considered as limited by the foregoing description.

What is claimed is:

1. A handle assembly for a medical device, comprising:
a handle body;
a first valve body housed at least partially within the handle body;
a second valve body housed at least partially within the handle body and positioned adjacent to the first valve body;
a lens wash inlet port extending from a first side the second valve body at a first angle with respect to a longitudinal axis of the second valve body; and
a lens wash outlet port extending from a second side of the second valve body at a second angle with respect to the longitudinal axis of the second valve body, wherein the first side of the second valve body faces a first direction and wherein the second side of the second valve body faces a second direction, and wherein the first direction is substantially opposite to the second direction.

2. The handle assembly of claim 1, further comprising:
an insufflation inlet port extending from the first side of the second valve body at a third angle with respect to the longitudinal axis of the second valve body; and
an insufflation outlet port extending from the second side of the second valve body at a fourth angle with respect to the longitudinal axis of the second valve body.

3. The handle assembly of claim 2, wherein the first angle, the second angle, the third angle, and the fourth angle are equal.

4. The handle assembly of claim 2, wherein the first angle, the second angle, the third angle, and the fourth angle are each approximately 45°.

5. The handle assembly of claim 2, wherein the first angle, the second angle, and the third angle are approximately equal, and wherein the fourth angle is different from each of the first angle, the second angle, and the third angle.

6. The handle assembly of claim 2, wherein each of the first angle, the second angle, and the third angle is approximately 90º, and wherein the fourth angle is approximately 45°.

7. The handle assembly of claim 2, wherein the lens wash outlet port extends toward a first end of the second valve body, and each of the insufflation outlet port, the insufflation inlet port, and the lens wash inlet port extend toward a second end of the second valve body.

8. The handle assembly of claim 2, wherein an interface between the insufflation outlet port and an inner chamber of the second valve body is positioned closer to a first end of the second valve body with respect to each of the insufflation inlet port, the lens wash inlet port, and the lens wash outlet port.

9. The handle assembly of claim 2, wherein an interface between the lens wash outlet port and a well of the second valve body is positioned closer to a first end of the second valve body with respect to the lens wash inlet port, and closer to a second end of the second valve body with respect to each of the insufflation inlet port and the insufflation outlet port.

10. The handle assembly of claim 1, wherein the first valve body includes a suction inlet port and a suction outlet port, and wherein the suction outlet port is positioned closer to a first end of the first valve body with respect to the suction inlet port.

11. The handle assembly of claim 10, wherein the suction outlet port extends towards the second valve body.

12. The handle assembly of claim 1, wherein the lens wash outlet port and the lens wash inlet port are collinear.

13. The handle assembly of claim 1, wherein a longitudinal axis of the first valve body is parallel to an axis of the handle body, and wherein the longitudinal axis of the second valve body is at an angle with respect to the axis of the handle body.

14. The handle assembly of claim 13, wherein the angle of the second valve body with respect to the axis of the handle body is in a range of 0° to 45°.

15. The handle assembly of claim 1, wherein each of the longitudinal axis of the second valve body and a longitudinal axis of the first valve body are parallel to an axis of the handle body.

16. A handle assembly for a medical device, comprising:
a handle body;
a first valve body housed at least partially within the handle body; and
a second valve body housed at least partially within the handle body and positioned adjacent to the first valve body,
wherein a longitudinal axis of the first valve body is parallel to an axis of the handle body, and
wherein a longitudinal axis of the second valve body is at an angle with respect to the longitudinal axis of the handle body.

17. The handle assembly of claim 16, wherein the angle of the longitudinal axis of the second valve body is in a range of 0° to 45°.

18. The handle assembly of claim 16, further comprising:
a lens wash inlet port extending from a first side the second valve body at a first angle with respect to a longitudinal axis of the second valve body;
a lens wash outlet port extending from a second side of the second valve body at a second angle with respect to the longitudinal axis of the second valve body;
an insufflation inlet port extending from the first side of the second valve body at a third angle with respect to the longitudinal axis of the second valve body; and
an insufflation outlet port extending from the second side of the second valve body at a fourth angle with respect to the longitudinal axis of the second valve body.

19. The handle assembly of claim 18, wherein the lens wash inlet port and the lens wash outlet port are collinear.

20. A handle assembly for a medical device, comprising:
a handle body;
a first valve body housed at least partially within the handle body;
a second valve body housed at least partially within the handle body and positioned adjacent to the first valve body;
a lens wash inlet port extending from a first side the second valve body, toward a first end of the second valve body and at a first angle with respect to a longitudinal axis of the second valve body; and
a lens wash outlet port extending from a second side of the second valve body, toward a second end of the second valve body and at a second angle with respect to the longitudinal axis of the second valve body,
wherein the first angle is equal to the second angle,
wherein a longitudinal axis of the first valve body is parallel to an axis of the handle body,
wherein a longitudinal axis of the second valve body is at an angle with respect to the longitudinal axis of the handle body, and
wherein the lens wash inlet port and the lens wash outlet port are collinear.
